(19) United States
(12) Patent Application Publication
(10) Pub. No.: US 2005/0241026 A1
(43) Pub. Date: Oct. 27, 2005

(54) Providing and communicating data message alerts stored on medical devices

(75) Inventors: James Esler, Coon Rapids, MN (US); Debra Coleman, Blaine, MN (US); Maureen McGuire, Roseville, MN (US); Christine Christie, St. Michael, MN (US); Richard Fears, Mounds View, MN (US); Chris Powers, Minneapolis, MN (US)

Correspondence Address: MERCHANT & GOULD PC, P.O. BOX 2903, MINNEAPOLIS, MN, 55402-0903, US

(73) Assignee: Cardiac Pacemakers, Inc.

(21) Appl. No.: 10/829,547

(22) Filed: Apr. 22, 2004

(51) Int. Cl. A61N 1/18

(52) U.S. Cl. D24/100

(57) ABSTRACT

Methods may include detecting whether a data message alert is stored in a dedicated alert field within a memory of the medical device upon interrogating the medical device with a programmer. The methods may also include communicating the data message alert via the programmer in response to detecting the data message alert stored in the dedicated alert field. A system may include a programmer, a medical device, and a link between the programmer and the medical device. The programmer is operative to provide, detect, and communicate a data message alert stored on the medical device in a dedicated alert field. The dedicated alert field is a free form data field having the capability to store the data message alert in any data format. The programmer is also operative to communicate the data message alert in any data format in which the data message alert is stored.

### TECHNICAL FIELD

The present invention relates generally to medical devices and programmers for medical devices. More particularly, the present invention is directed to providing, detecting, and communicating message alerts stored on one or more medical devices.

### BACKGROUND

Medical devices, including pulse generators such as pacemakers and implantable cardiac defibrillators, are frequently involved in activities that call for the sharing of pertinent information regarding the medical device and/or the patient utilizing the medical device. For instance, in clinical studies, it is important to track how a medical device is programmed so as to be able to ascertain the effectiveness of the therapy applied. Previous methods and systems for sharing information included recording information on a clipboard, a chart for a patient, or in a database used to store other clinical study data. These previous methods and systems require a clinician or physician to actively look up information concerning whether a given medical device is enrolled in a clinical study. Thus, information could be easily overlooked by a clinician or physician focused on interfacing with the medical device, especially in emergency situations. The physician or clinician employs a device programmer to adjust performance parameters of the medical device, such as an implantable pulse generator. Consequently, when the programming of such a device is modified, there is the likelihood that the modifications will not be recorded in the study protocol if the physician misses the fact that the device is enrolled in a clinical study. Such unrecorded programming modifications may in turn significantly impact the accuracy of the study results or even invalidate the study results for a particular device.

Further, there is the likelihood that any future programming may be conducted without knowledge of special clinical study programming instructions. For instance, certain parameter settings of the medical device may need to be maintained in order to avoid adverse events associated with the clinical study. Unorthodox programming of a medical device enrolled in a clinical study may also jeopardize the accuracy and validation of the study.

There are also financial incentives that motivate study sites to protect patient enrollment in a study. The missed opportunity of capturing an event endangers the patient's continued enrollment at the expense of the patient's study site.

Other example activities for medical devices where the sharing of information is necessary may include, but are not limited to, a change in the drug regime of a patient utilizing a medical device, information regarding suspect issues with the medical device or the components of the medical device, and even a reminder to administer certain medical procedures to the patient. Entrusting the sharing of such important information to previous methods and systems increases the risk of missing the intended communication.

It is with respect to these and other considerations that the present invention has been made.

### SUMMARY

In accordance with the present invention, the above and other problems are solved by embodiments of the present invention. Embodiments of the present invention are directed to methods, systems, computer program products, and propagated signals for providing and communicating data message alerts stored on a medical device. The present invention allows for efficient, consistent, and user-friendly organization and reorganization of patient and medical device activity. The present invention addresses problems such as, but not limited to, those mentioned above by providing and communicating a marker, alert, or notification stored within the medical device to prompt a clinician/physician of patient and or device specific activity. Additionally, due to the present invention, multiple clinicians or physicians may interface with a medical device via a programmer and each clinician/physician is alerted with regard to patient and/or medical device specific activity. Thus, the present invention reduces the likelihood of not recording or noticing modifications to medical devices or a patient care regime.

One embodiment of the present invention is a method for providing and communicating data message alerts stored on a medical device. The method involves interrogating the medical device with a programmer. Upon interrogating the medical device, the programmer detects whether a data message alert is stored in a dedicated alert field within a memory of the medical device. It should be appreciated that the data message alert originates from outside the medical device. When the programmer detects a stored data message alert in dedicated alert field of the medical device, the programmer may communicate the data message alert to a user of the programmer via a display, a printer, an audio output device, and/or multi-media output.

Another embodiment of the present invention is a computer program product including a computer usable medium storing control logic for causing a computer to provide and communicate data message alerts stored on a medical device. The control logic includes computer readable program code for causing the computer to interrogate the medical device. Upon interrogating the medical device, the computer detects whether a data message alert, originating from outside the medical device, is stored in a dedicated alert field within the memory of the medical device. In response to detecting the data message alert stored in the dedicated alert field, the computer communicates the data message alert in graphics, video, text, animation, and or sound data formats.

Still another embodiment of the present invention is a propagated signal on a carrier that is detectable by a computing system and that encodes data and a computer program of instructions for executing a computer process for providing and communicating data message alerts stored on a medical device. The computer process involves receiving a data message alert at a medical device from outside the medical device via a computing device, such as from a programmer computing device. Once the data message alert is received, the data message alert is saved to a dedicated alert field of the medical device. The computer process also involves interrogating the medical device. Upon interrogating the medical device, the computer process involves detecting whether the data message alert is stored in the dedicated alert field of the medical device. The computer process also involves communicating the data message alert in response to detecting the data message alert stored in the dedicated alert field.

Additionally, another embodiment of the present invention is a system for providing and communicating data message alerts stored on a medical device. The system includes a computing device, such as a programmer, a medical device, such as an implantable pulse generator, and a link between the computing device and the medical device. The computing device is operative to provide, detect, and/or communicate a data message alert stored on the medical device. The medical device stores the data message alert in a dedicated alert field after receiving the data message alert from outside the medical device. For instance, the medical device may receive the data message alert from a programmer computing device. The dedicated alert field is a free form data field having the capability to store a data message alert in any data format including text, video, audio and graphics file formats. The computing device is operative to interrogate the medical device and thereby detect whether the data message alert is stored in the dedicated alert field. Also, in response to detecting the data message alert stored in the dedicated alert field, the computing device is operative to communicate the data message alert in any data format in which the data message alert is stored.

As described, the invention may be implemented as a computer process, a computing system or as an article of manufacture such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process.

These and various other features as well as advantages, which characterize the present invention, will be apparent from a reading of the following detailed description and a review of the associated drawings.

### DESCRIPTION OF THE DRAWINGS

FIG. 1 is a schematic drawing illustrating components and a screen display of an embodiment of the present invention and an environment in which the embodiment is used.

FIG. 2 illustrates a computing system architecture for a computing device incorporating input and output devices utilized in embodiments of the present invention.

FIG. 3 illustrates an operational flow performed in providing a data message alert in an embodiment of the present invention.

FIG. 4 illustrates an operational flow performed in providing and communicating a data message alert in another embodiment of the present invention.

FIG. 5 illustrates an operational flow performed in processing a data message alert for remote storage and/or remote communication in another embodiment of the present invention.

### DETAILED DESCRIPTION

As described briefly above, embodiments of the present invention provide methods, computer program products, propagated signals, and systems for providing and communicating data message alerts stored on medical devices. Providing and communicating data message alerts reduces the likelihood that important information will be overlooked when programming or interfacing with medical devices. In the following detailed description, references are made to accompanying drawings that form a part hereof, and in which are shown by way of illustration specific embodiments or examples. These embodiments may be combined, other embodiments may be utilized, and structural changes may be made without departing from the spirit and scope of the present invention. The following detailed description is, therefore, not to be taken in a limiting sense, and the scope of the present invention is defined by the appended claims and their equivalents. In this document “and/or” refers to non-exclusive “or” (e.g., “A and/or B” includes each of “A but not B.” “B but not A,” and “A and B”).

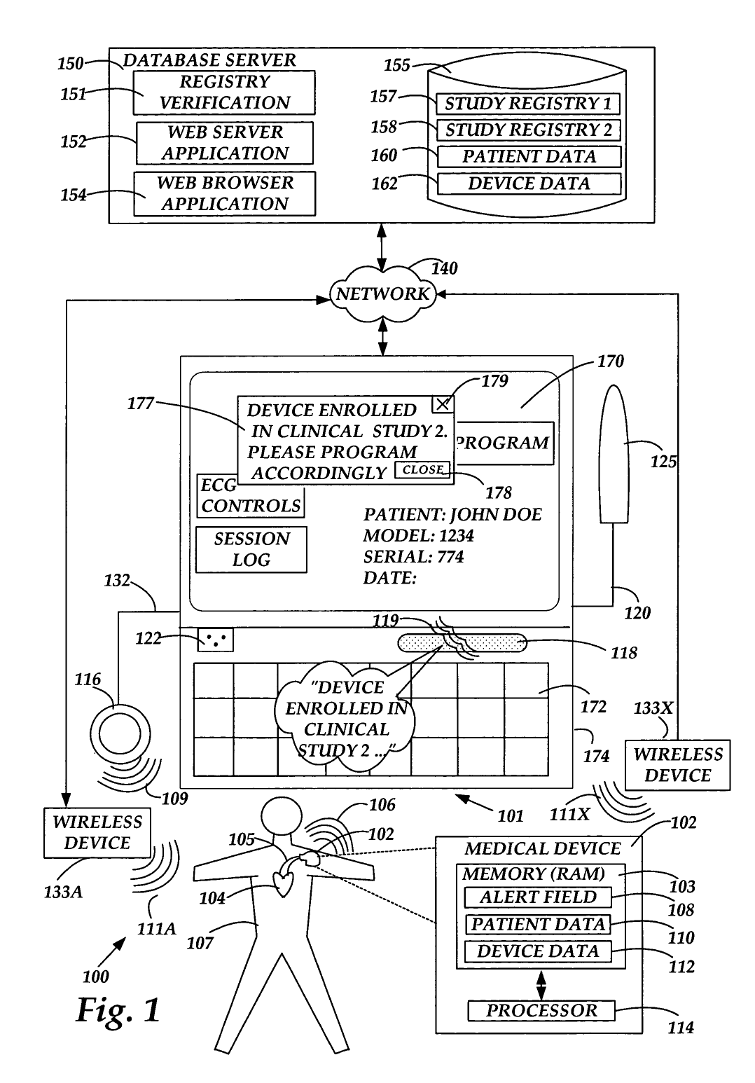

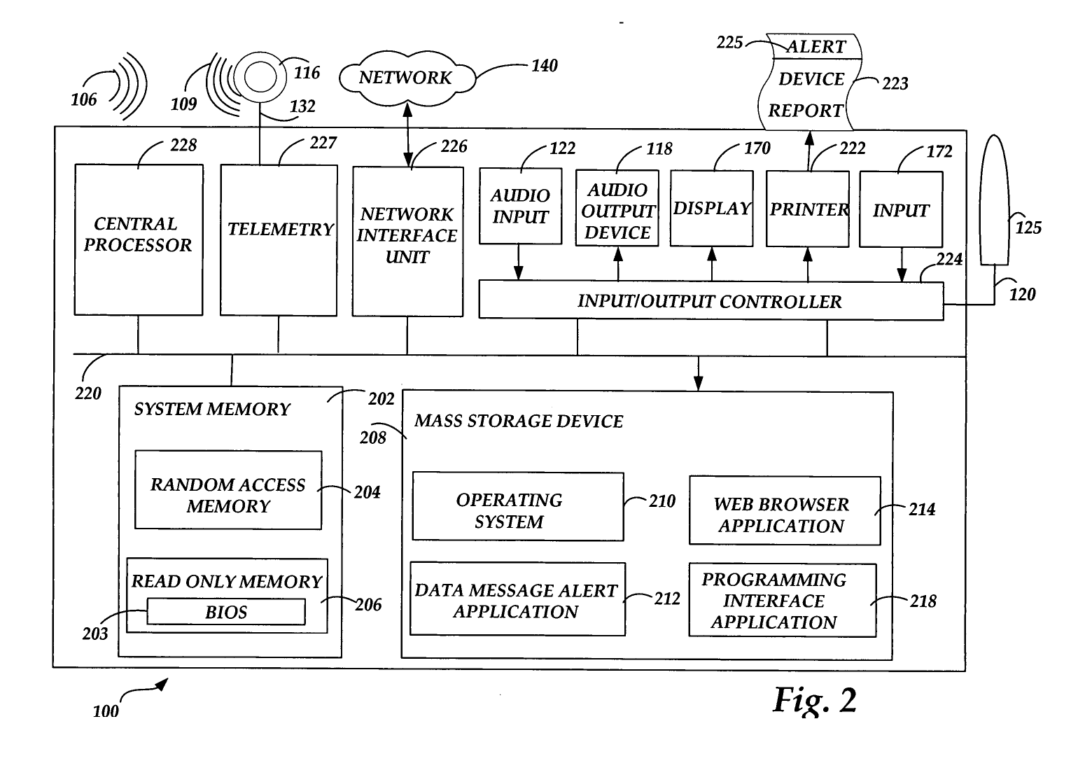

Referring now to the drawings, in which like numerals represent like elements through the several figures, aspects of the present invention and the exemplary operating environment will be described. FIGS. 1-2 and the following discussion are intended to provide a brief, general description of a suitable computing environment in which the embodiments of the invention may be implemented. While the invention will be described in the general context of program modules that execute to provide and communicate data message alerts stored on medical devices, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

The present embodiments of the invention will be described in applications involving implantable medical devices including, but not limited to, implantable cardiac rhythm management systems such as pacemakers, defibrillators, and biventricular or other multi-site coordination devices. However it is understood that the present methods, systems, computer program products, and propagated signals may be employed in un-implanted devices, including, but not limited to, external pacemakers, cardioverter/defibrillators, pacer/defibrillators, biventricular or other multi-site coordination devices, monitors, programmers, and recorders.

Referring now to FIG. 1, a system 100 for providing and communicating data message alerts stored on a medical device will be described. As shown in FIG. 1, the system includes a programmer 101 and a medical device 102, such as an implantable pulse generator. The medical device 102 is implanted in a patient 107 and coupled to the heart 104 of the patient 107 by one or more leads 105. The programmer 101 is adapted to be communicatively coupled to the medical device 102 to receive and transmit control commands, program instructions, and cardiac data to and from the medical device 102 via an antenna 116 that radiates a signal 109 and receives a signal 106 produced by the medical device 102. The programmer 101 is used in application to various activities such as electrical lead 105 placement and implantable device 102 optimization.

The medical device 102 generally includes a memory 103, such as a random access memory (RAM), and a processor 114 such as for storing and processing instructions received from the loop antenna 116. The memory 103 includes a dedicated alert field 108 designated to store data message alerts that are also received via the loop antenna 116. The dedicated alert field 108 may comprise an array of bytes in RAM and is a free form field capable of storing data message alerts in any data format. The data message alerts and data formats may include, but are not limited to, ASCII text, multimedia such as MPEG, graphic file formats such as JPEG, GIF, and PNG, audio and/or audio encoding schema, and XML and/or XML schema definition. The memory 103 may also include a patient data storage area 110 for storing patient data and a device data storage area 112 for storing information pertaining to the medical device 102.

The programmer 101 comprises a standard personal or laptop computing device operative to execute a data message alert application, which executes in response to user initiation or in conjunction with the process of interrogating medical devices. FIG. 1 shows an external view of the programmer 101 according to an embodiment of the present invention. As briefly described, the programmer 101 includes the antenna 116 for sending and receiving modulated electromagnetic signals that may establish bi-directional communications with the medical device 102. The loop antenna 116 radiates electromagnetic energy in the form of the propagated signal 109. The signal 109 generally has encoded information such as a data message alert, instructions for the medical device 102, and/or trending data to be stored by the medical device 102. The medical device 102 receives the signal 109 from the loop antenna 116 and the processor 114 interprets the encoded information and/or carries out the instruction.

The programmer 101 that is shown includes an input/output controller 224 (see FIG. 2) interfaced with a number of input devices. The input devices include, but are not limited to, a keyboard 172, an audio input 122, and a stylus 125, that allow the user to input information such as data message alerts and function selections. The stylus 125 communicates with the input/output controller 224 through line 120. Alternatively, the programmer 101 may also include a “mouse”—type pointing device rather than a stylus. The programmer 101 may be enclosed within a housing 174 made of metal, plastic, or other rigid material. The keyboard 172 and display screen 170 may be integrated into the housing 174 such that the programmer 101 is enclosed within a single housing. Alternatively, multiple housings may be provided for various components.

The programmer 101 also includes display screen 170 and an audio output device 118 controlled by the input/output controller 224. The display screen 170 may serve as an input and output device, which is provided with touch-sensitive capability such that a user can interact with the programmer 101 by touching the display area on the display screen 170 with a detached stylus (not shown), or even the user's finger. The display screen 170 may be a liquid crystal display (LCD) or other display type such as a cathode ray tube (CRT). The display screen 170 may show various forms of information, such as programmer menus, device parameter settings, and any information sent or received via the antenna 116 including data message alerts.

Data message alerts may be communicated via the display screen 170 in the form of a pop-up window 177 including text, video, animation, and/or graphics utilized in conveying the data message alert to a user of the programmer 101. As shown, the data message alert comprises a text message displayed via the pop-up window 177 and providing notice that the medical device 102 is enrolled in a clinical study and that special programming of the device may be necessary. The data message alert may be acknowledged by selecting the close button 178 or the close button 179 of the data message alert pop-up window 177. Data message alerts may also be communicated via the audio output device 118 in the form of a voice or audio message alert acoustic signal 119. The audio output device 118 may be used in conjunction with multi-media communication of the data message alert or as a means to convey a voice or audio data message alert.

Data message alerts originate from outside the medical device and have a plurality of uses. For example, a data message alert may include a message communicating that the medical device 102 and /or the patient 107 utilizing the medical device are enrolled in a clinical study. This use could prevent improper programming practices outside the guidelines of the clinical study and/or serve as a reminder to record all modifications. Secondly, a data message alert may include a message communicating a drug regime for the patient 107 utilizing the medical device 102. A data message alert may also include a message from one clinician, communicating information concerning a component of the medical device, to another clinician subsequently interrogating the medical device. This use could warn a clinician of a suspect electrical lead 105 or other component of the medical device 102. A data message alert may also include a message communicating a reminder to send in a product registration for the medical device 102.

The system 100 may also include a database server 150 operative to receive uploads from the programmer 101 via the network 140, such as the Internet. The database server 150 may include a registry verification application 151 for verifying whether the correct data is stored in the proper registry, a web server application 152, and a web browser application 154 such as INTERNET EXPLORER from MICROSOFT CORPORATION of Redmond, Wash., and a database 155 for storing the uploaded data. For instance, information in the database may be stored according to a clinical study with which the data is associated. Thus, data associated with clinical study one (1) is stored in a study registry 157 for data associated with clinical study one. Similarly, data uploaded to the database 155 and associated with a clinical study two (2) is stored in a study registry 158. Uploaded data may also be stored in a patient data registry 160 according to patient association and/or in a device data registry 162 according to medical device association. The registry verification application 151 may execute to facilitate the proper storage of data.

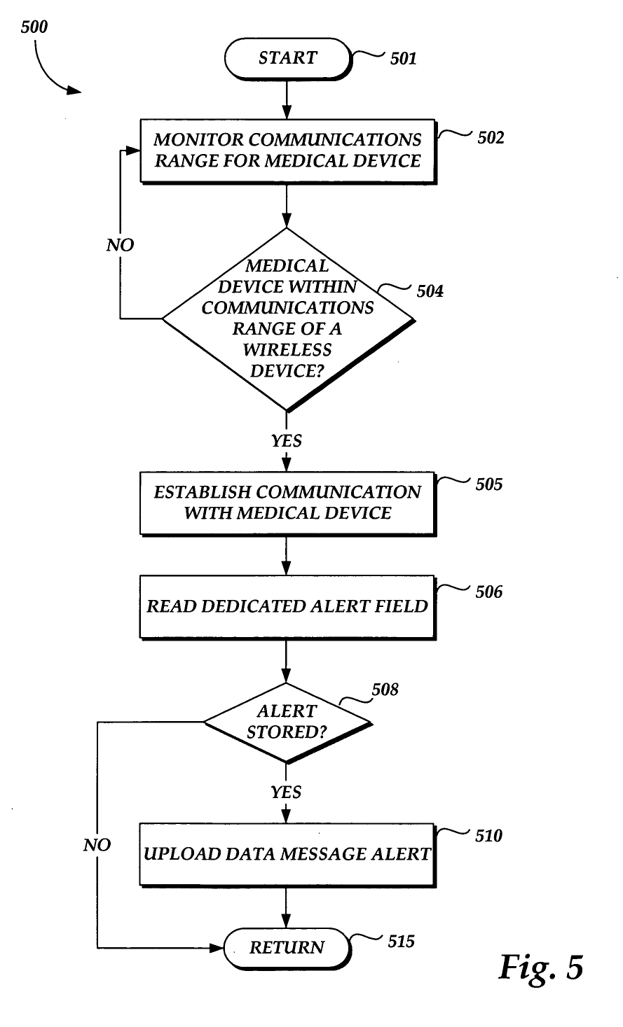

Additionally, the system 100 may include one or more wireless devices 133A-133X. The wireless devices 133A-133X comprise transceivers operative to interrogate the medical device 102 in response to the medical device 102 entering a communications range of any wireless device 133A-133X represented by a radiated signals 111A-111X. For instance, the wireless devices may be disbursed throughout a patient's home and when the patient 107, with the medical device 102, enters the communications range of any wireless device 133A-133X, the wireless device monitoring the communications range entered interrogates the medical device 102. Upon interrogating the medical device 102, the wireless device 133A-133X detects whether a data message alert is stored in the alert field 108. If a data message alert is detected, the wireless device is operative to upload the data message alert to the database 155 and/or the programmer 101 via the network 140. Additional details regarding data message alert uploads via the wireless devices 133A-133X will be described below with respect to FIG. 5.

FIG. 2 shows a block diagram of the programmer 101 computing device incorporating input and output communication functions. The programmer 101 includes a communications device such as a telemetry module 227, a central processor 228, a system memory 202, and a system bus 220 that couples the system memory 202 to the central processor 228. The system memory 202 includes read-only memory (ROM) 206 and random access memory (RAM) 204. A basic input/output system 203 (BIOS), containing the basic routines that help to transfer information between elements within the programmer 101, such as during start-up, is stored in ROM 206. The programmer 101 further includes a mass storage device (MSD) 208 for storing an operating system 210, a programming interface application 218, and other application programs such as a web browser application 214 for example INTERNET EXPLORER from MICROSOFT CORPORATION of Redmond, Wash. The MSD 208 may also store a data message alert application 212 which in cooperation with the processor 228 is operative to provide and communicate data message alerts stored on medical devices.

The MSD 208 is connected to the central processor 228 through a mass storage controller (not shown) connected to the system bus 220. The MSD 208 and its associated computer-readable media, provide non-volatile storage for the programmer 101. Although the description of computer-readable media contained herein refers to a mass storage device, such as a hard disk or CD-ROM drive, it should be appreciated by those skilled in the art that computer-readable media can be any available media that can be accessed by the CPU 210.

Telemetry module 227 receives signals from and sends signals to the central processor 228 through the signal bus 220. Telemetry module 227 also sends to and receives signals from the loop antenna 116, which typically is a wire loop. The telemetry communications device 227 may use circuitry such as that known in the art for implantable device communications.

The medical device 102 radiates a signal 106 that also has encoded information, such as a data message alert being extracted and communicated from the dedicated alert field 108. The radiated signal 106 propagated from the medical device 102 is received by the loop antenna 116 and is converted to an electrical signal that is transferred to the telemetry module 227. The telemetry module 227 may then employ an analog-to-digital conversion to convert the received signal to a data signal that is then passed to the central processor 228. Alternatively, or in addition to feeding received signals to the processor 228, the telemetry module 227 may feed signals directly to the display device 170 for real-time display of the information encoded on the signal 106 such as a data message alert.

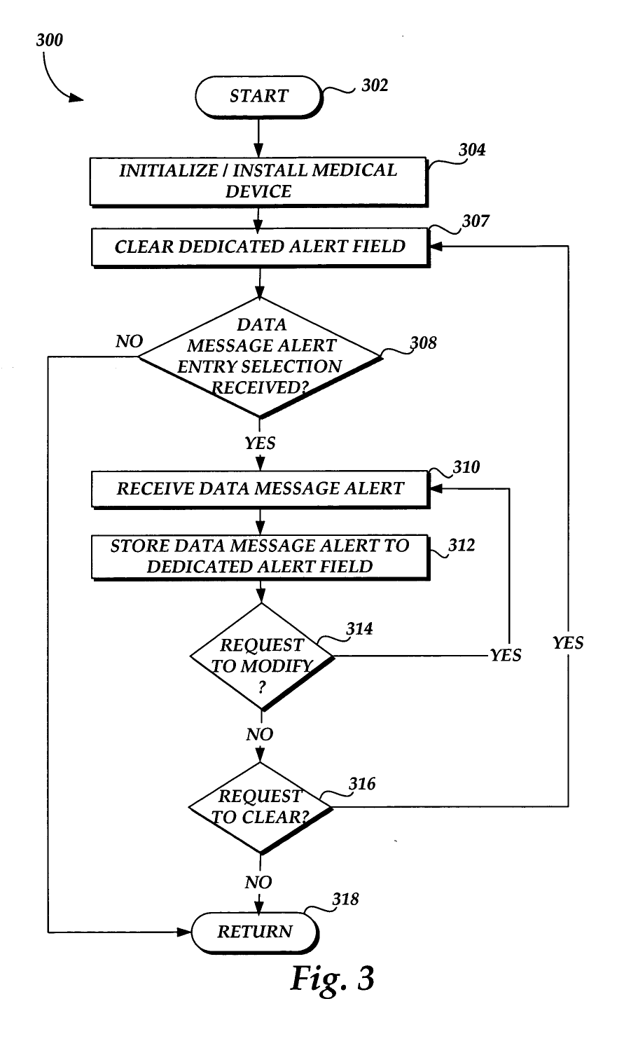

The central processor 228 may employ various operations, discussed in more detail below with reference to FIGS. 3, 4, and 5 to provide and utilize the signals propagated between the programmer 101 and the communications device 227. The processor 228 may store data to and access data from mass storage device 208, such as electronic memory or magnetic storage. Data is transferred to and received from the storage device 208 through the system bus 220. The processor 228 may be a general-purpose computer processor or processor typically used for a programmer. Furthermore as mentioned below, the processor 228, in addition to being a general-purpose programmable processor, may be firmware, hard-wired logic, analog circuitry, other special purpose circuitry, or any combination thereof.

The processor 228 may also transfer a display signal to the display device 170 through the system bus 220. The display signal may include an information signal produced by the communication device 227. The information signal component from the communications device 140 may encode the data message alert received from the medical device 102. The display device 170 then displays a representation of the data message alert in the form of a pop-up window as illustrated by the pop-up window 177 referenced in FIG. 1 discussed above.

The input/output controller 224 may also be included with the programmer 101 for receiving and processing input from a number of input devices, including the keyboard 172, the audio and/or voice input 122, the stylus 125 and mouse (not shown). Similarly, the input/output controller 224 may provide output to the display screen 170, a printer 222, the audio output device 118, and/or other type of output devices. The input/output controller 224 communicates with the processor 228 through the system bus 220. The printer 222 may produce a data message alert in the form of a paper copy or a printed report 223. A data message alert 225 may be included in the header of each printed medical device report 223 until the data message alert 225 is removed from the dedicated alert field 108. The printer 222 receives the data signal for the paper copy through system bus 220 and the input/output controller 224.

According to various embodiments of the invention, the programmer 101 operates in a networked environment, as shown in FIG. 2, using logical connections to remote computers through the network 140, such as the Internet, an Intranet, or a local area network (LAN). The programmer 101 may connect to the network 140 via a network interface unit 226 connected to the system bus 220. It should be appreciated that the network interface unit 226 may also be utilized to connect to other types of networks and remote computer systems. The network interface unit 226 may also serve as the interface for receiving data message alerts over the network 140.

A computing device, such as the programmer 101, typically includes at least some form of computer-readable media. Computer readable media can be any available media that can be accessed by the computing system 101. By way of example, and not limitation, computer-readable media might comprise computer storage media and communication media.

Computer storage media includes volatile and nonvolatile, removable and non-removable media implemented in any method or technology for storage of information such as computer readable instructions, data structures, program modules or other data. Computer storage media includes, but is not limited to, RAM, ROM, EPROM, flash memory or other memory technology, CD-ROM, digital versatile disks (DVD) or other optical storage, magnetic cassettes, magnetic tape, magnetic disk storage or other magnetic storage devices, or any other medium that can be used to store the desired information and that can be accessed by the computing system 101.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, and other wireless media. Combinations of any of the above should also be included within the scope of computer-readable media. Computer-readable media may also be referred to as computer program product.

FIG. 3 illustrates an operational flow 300 performed in providing and storing a data message alert upon initialization and/or installation of the medical device 102 in an embodiment of the present invention. The operational flow 300 begins at start operation 302 then continues to initialize/install operation 304 where the programmer establishes a telemetric link with the medical device 102 via the signal 109 and the medical device 102 is implanted. Programming instructions, including operating values, are also sent to the processor 114 within the medical device 102 as part of the initialization and installation process. Also, upon initializing the medical device 102, the dedicated alert field 108 is cleared of any data message alerts at clear operation 307. Thus, in the present embodiment, the medical device is initialized to be clear of data message alerts with the option to enter a new data message alert.

The operational flow 300 then continues to detect operation 308 where a determination is made as to whether a data message alert entry selection has been received from a user desiring to enter a data message alert. The display screen 170 may provide a user interface that allows the user to make selections from menus. An initialization entry menu may be provided to display entry options that a user can select to enter data or programming instructions. The user may select to enter the data message alert application 212. Using at least one input device which may include, but is not limited to, a keyboard 172, an audio input device, and or a stylus 125, the user may choose to enter a data message alert upon initialization and installation of the medical device 102. When at detect operation 308 a data message alert entry selection is not received, the operational flow 300 returns control to other routines at return operation 318.

When at detect operation 308, a data message alert entry selection is received, the operational flow 300 continues to receive operation 310. At receive operation 310 a data message alert is received from a user via at least one input device. For instance, the user may input the data message alert as text via use of the keyboard 172. The user may also record the data message alert as an audio message via the audio input device 122. After receiving the data message alert, the operational flow 300 continues to save operation 312 where the received data message alert is saved to the dedicated alert field 108 within the memory 103 of the medical device 102.

It should be appreciated that the dedicated alert field 108 is of a free form such that the data message alert may be stored and communicated in any data format. For example, if the format of the data message alert is an audio encoding schema, the dedicated alert field 108 is capable of storing and the programmer 101 is capable of communicating the data message alert in an audio encoding schema format. In contrast, if the format of the data message alert is a multimedia format, the dedicated alert field 108 is capable of storing and the programmer is capable of communicating the data message alert in a multimedia format.

Once the data message alert has been saved to the dedicated alert field 108, the operational flow continues to detect operation 314 where determination is made as to whether a request to modify the data message alert has been received. A user has the option to modify the data message alert. When a request to modify has been received, the operational flow 300 branches back to receive operation 310. If a request to modify is not detected, the operational flow 300 continues from detect operation 314 to detect operation 316.

At detect operation 316 a determination is made as to whether a request to clear the dedicated alert field has been received. A user has the option to clear the data message alert. When a request to clear has been received, the operational flow 300 branches back to clear operation 307. If a request to clear is not detected, the operational flow continues from detect operation 316 to return operation 318 where control is returned to other routines.

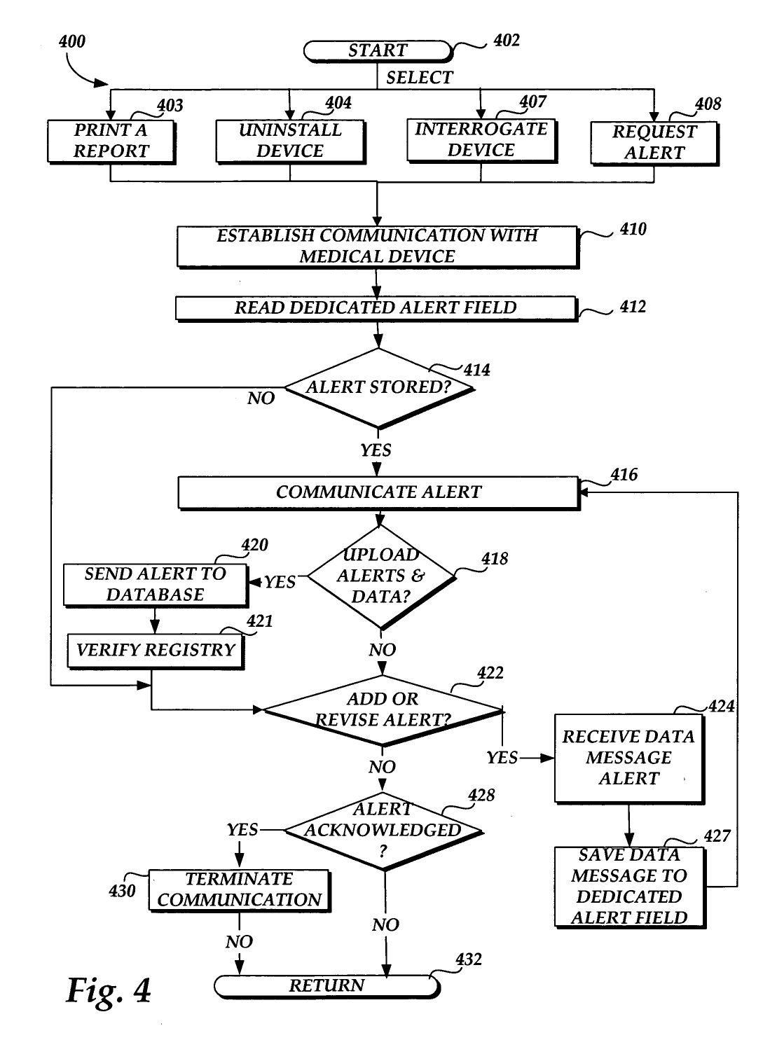

FIG. 4 illustrates an operational flow 400 performed in providing and communicating a data message alert in another embodiment of the present invention. The operational flow 400 begins at start operation 402 and continues to at least one of four operations. The processor 228 may receive a selection from a user via a menu of programmer 101 options. The programmer 101 options may include, but are not limited to, a print operation 403, an uninstall operation 404, an interrogate operation 407, and a request operation 408. For instance, selection of the print operation 403 initiates the process of printing a report concerning the medical device. The data message alert application 212 is operative to communicate the data message alert in the form of printed text on every printed report 223 generated by the programmer 101 while the data message alert is stored. Also, selection of the uninstall operation 404 initiates the process for removing medical devices, such as explanting the implanted medical device 102. Third, selection of the interrogate operation 407 initiates the process for interrogating the medical device 102 for patient and/or medical device specific information. Last, selection of the request operation 408 specifically initiates detection and communication of any data message alert stored on the medical device 102. In the alternative, it should be appreciated that the wireless device 133 may execute passive interrogation of the medical device 102 in response to the medical device 102 being within a communications range of any wireless device 133A-133X. Additional details regarding uploading a data message alert upon passive interrogation of the medical device will be described below with respect to FIG. 5.

In response to the selection of at least one of the four programmer 101 options, the operational flow 400 continues to communication operation 410 where the programmer 101 establishes communication with the medical device 102 via the telemetry module 227 and the loop antenna 116. The operational flow 400 then continues to read operation 412 where the programmer reads the dedicated alert field of the medical device 102. Next at detect operation 414 the programmer 101 determines whether a data message alert is stored in the dedicated alert field 108. In response to no data message alert being stored, thus having a null value in the dedicated alert field 108, the operational flow 400 continues to detect operation 422 described below. Upon detecting a data message alert in the dedicated alert field 108 at detect operation 414, the operational flow 400 continues from detect operation 414 to communicate operation 416.

At communicate operation 416, the programmer communicates the data message alert via one or more output devices of the programmer. For instance, if the data message alert stored is an audio recording, the programmer plays the audio recording via the audio output device 118. Also, if the data message alert is a text message, the programmer 101 displays the text message in a pop-up window via the display 122. It should be appreciated that communication of the data message alert may persist until acknowledged by the user thereby assuring notice to the user of the data message alert.

Next, the operational flow 400 continues to detect operation 418 where a determination is made as to whether the data message alerts and patient data are to be uploaded to a database 150 for storage in an associated storage location. If the data message alerts are not to be uploaded, the operational flow 400 continues to operation 422 described below. If the data message alerts and or the patient data are uploaded to the database 157, the operational flow 400 continues to send operation 420. At send operation 420, the programmer sends to data message alert and or patient data to the database 150. For example, in a clinical study, patient data is grouped by clinical study. Thus, when a data message alert providing a reminder that a patient and the medical device 102 are enrolled in a clinical study is uploaded to the database 150, the data message alert is stored in a study registry associated with the study. For instance, if the study is clinical study one (1), the data message alert and associated patient data will be uploaded to study registry 157 as shown in FIG. 1.

Next the operational flow 400 continues to verify operation 421 where the processor 228 executing the data message alert application 212 verifies whether the upload is being sent to the correct study registry. Thus, as the patient data is being uploaded, the data message alert application 212 and the registry verification application 151 execute to provide a real time verification as to whether the upload is being sent to the correct study site thereby providing the site with immediate feedback. The registry verification application 151 may read the clinical study identified in the data message alert and or a serial number of the medical device 102 associated with the data in order to verify proper storage.

Then operational flow 400 then continues to detect operation 422 where a determination is made as to whether a user has selected to add or revise a data message alert. When a user selects to add or revise a data message alert, the operational flow 400 continues to receive operation 424 where the new or revised data message alert is received. Next at save operation 427, the new or revised data message alert is saved to the dedicated alert field of the medical device 102. Then the operational flow 440 branches back from save operation 427 to communicate operation 416 described above.

If at detect operation 422, a user has not selected to add or revise a data message alert, the operational flow 400 continues to acknowledge operation 428. At acknowledge operation 428 a determination is made as to whether the data message alert has been acknowledged. For instance, the data message alert may be acknowledged by selecting the close button 178 or the close button 179 of the data message alert pop-up window 177. If the data message alert is not acknowledged, communication of the data message alert persists and the operational flow 400 returns control to other routines at return operation 432. If the data message alert is acknowledged, the operational flow continues from acknowledge operation 428 to terminate operation 430 where communication of the data message alert is terminated until another interrogation with the medical device occurs. Control is then returned to other routines at return operation 432.

FIG. 5 illustrates an operational flow 500 performed in processing a data message alert for storage and/or communication in another embodiment of the present invention. The operational flow 500 begins at start operation 501 and continues to monitor operation 502. At monitor operation 502, the wireless devices 133A-133X monitor respective communication ranges to detect entry of the medical device 102. The wireless devices 133A-133X may be placed throughout the home of a patient to periodically interrogate the medical device 102 for updates on the patient's condition. Next, the operational flow 500 continues to detect operation 504.

At detect operation 502, a determination is made as to whether the medical device 102 is within a communications range of any wireless device 133A-133X. If the medical device 102 is not within a communications range of any wireless devices 133A-133X the operational flow 500 branches back to monitor operation 502 described above. If the medical device 102 is within a communications range of any wireless device, such as wireless device 133A, the operational flow continues to communication operation 505.

At communication operation 505, the wireless device 133A establishes communication with the medical device 102. The wireless devices may include a transceiver for receiving and transmitting wireless signals. The operational flow 500 then continues to read operation 506 where the wireless device 133A reads the dedicated alert field 108 of the medical device 102. Next at detect operation 508 the wireless device 133A determines whether a data message alert is stored in the dedicated alert field 108. In response to no data message alert being stored, thus having a null value in the dedicated alert field 108, the operational flow 500 returns control to other routines at return operation 515.

Upon detecting a data message alert in the dedicated alert field 108 at detect operation 508, the operational flow 500 continues from detect operation 508 to upload operation 510. At upload operation 510, the wireless device uploads the data message alert via the network 140 to the database 150 where the data message alert is stored. The data message alert is also uploaded to any computing device, such as the programmer 101, connected to the network 140 and authorized to communicate the data message alert. The operational flow 500 then returns control to other routines at return operation 515.

The various embodiments of the operations of the invention, such as but not limited to those of FIGS. 3, 4, and 5, are implemented as logical operations in the computing system. The logical operations are implemented (1) as a sequence of computer implemented acts or program modules running on a computing system of the programmer 101 including a processing module such as processor 228 and/or (2) as interconnected machine logic circuits or circuit modules within the computing system.

This implementation is a matter of choice dependent on the performance requirements of the computing system implementing the invention. Accordingly, the logical operations making up the embodiments of the invention described herein are referred to as operations, structural devices, acts or modules. It will be recognized by one of ordinary skill in the art that various changes in the form and details may be made and that the operations, structural devices, acts or modules may be implemented in software, in firmware, in special purpose digital logic, analog circuits, and any combination thereof without deviating from the spirit and scope of the present invention as recited within the claims attached hereto.

1. A method for providing and communicating data message alerts stored on a medical device, the method comprising:
interrogating the medical device with a computing device; and
upon interrogating the medical device, communicating via the computing device, a data message alert stored within a memory of the medical device wherein the data message alert originates from outside the medical device. (OR wherein the DMA does not originate within the medical device)

2. The method of claim 1, further comprising:
detecting whether the data message alert is stored within the memory of the medical device wherein the data message alert is communicated in response to detecting the data message alert stored within the memory of the medical device.

3. The method of claim 2, wherein the data message alert is stored in a dedicated alert field within the memory of the medical device.

4. The method of claim 1, further comprising:
receiving a new data message alert; and
in response to receiving the new data message alert, saving the new data message alert to the memory as the data message alert.

5. The method of claim 1, further comprising:
receiving a revised data message alert; and
in response to receiving the revised data message alert, saving the revised data message alert to the memory as the data message alert.

6. The method of claim 1, prior to interrogating the medical device, further comprising at least one of initializing and installing the medical device.

7. The method of claim 6, wherein initializing the medical device includes at least one of the following:
clearing the memory of any data message alerts; and
entering a new data message alert.

8. The method of claim 6, wherein installing the medical device comprises implanting the medical device in a patient.

9. The method of claim 6, further comprising:
uninstalling the medical device; and
in response to uninstalling the medical device, interrogating the medical device.

10. The method of claim 9, wherein uninstalling the medical device comprises explanting the medical device from the patient.

11. The method of claim 1, wherein the memory of the medical device comprises a random access memory (RAM).

12. The method of claim 1, further comprising:
requesting the data message alert; and
in response to requesting the data message alert, interrogating the medical device.

13. The method of claim 3, wherein interrogating the medical device comprises:
establishing communication with the medical device; and
reading the dedicated alert field.

14. The method of claim 2, further comprising, in response to detecting the data message alert stored, uploading the data message alert to a database.

15. The method of claim 1, wherein communicating the data message alert via the computing device comprises communicating the data message alert in at least one of a variety of data formats compatible for storage in the memory.

16. The method of claim 15, wherein at least one of the data message alert and the variety of data formats compatible for storage in the memory include at least one of the following data formats:
ASCII text;
multi-media;
audio;
audio encoding schema;
XML; and
XML schema definition.

17. The method of claim 16, wherein communicating the data message alert comprises at least one of the following:
displaying a text pop-up window containing a text message alert via a display device of the computing device;
displaying and playing a pop-up multi-media message alert via the display device and an audio output device of the computing device;
playing an audio message alert via the audio output device of the computing device; and
displaying a text pop-up window containing an XML text string message alert via the display device of the computing device.

18. The method of claim 4, wherein receiving a new data message alert comprises at least one of the following:
receiving text of the new data message alert via a first input device of the computing device;
receiving a multi-media recording of the new data message alert via a second input device of the programmer;
receiving an audio recording of the new data message alert via one of the second input device and a third input device of the computing device; and
receiving an XML text string of the new data message alert via one of the first input device and the second input device of the computing device.

19. The method of claim 3, wherein detecting whether a data message alert is stored in the memory comprises detecting whether the dedicated alert field is null.

20. The method of claim 1, further comprising:
receiving an acknowledgement of the data message alert communicated; and
in response to receiving the acknowledgement, terminating communication of the data message alert.

21. The method of claim 3, further comprising:
receiving a request to clear the data message alert from the dedicated alert field; and
in response to receiving the request to clear, clearing the data message alert from the dedicated alert field whereby the dedicated alert field is rendered null.

22. The method of claim 1, wherein the data message alert comprises at least one of patient-specific information and medical device-specific information.

23. The method of claim 14, wherein the data message alert comprises at least one of the following:
a message communicating that at least one of the medical device and a patient utilizing the medical device are enrolled in a clinical study;
a message communicating a drug regime for the patient utilizing the medical device;
a message communicating information concerning a component of the medical device; and
a message communicating a reminder to send in a product registration for the medical device.

24. The method of claim 23, wherein the data message alert comprises the message communicating that at least one of the medical device and the patient utilizing the medical device are enrolled in the clinical study and wherein the method further comprises:
uploading at least one of patient data and medical device data to the database; and
utilizing the data message alert to verify that at least one of the patient data and the medical device data are being uploaded to a correct study registry in the database for the clinical study.

25. The method of claim 19, further comprising in response to detecting that the dedicated alert field is not null, including the data message alert in any reports generated by the programmer until the dedicated alert field is rendered null.

26. The method of claim 25, wherein the data message alert comprises a text message alert and wherein including the data message alert in the any reports generated by the programmer comprises printing the text message alert in any printouts generated by the programmer.

27. The method of claim 26, wherein the text message alert is printed as header text of the any printouts generated by the programmer.

28. The method of claim 1, further comprising:
interrogating the medical device with at least one wireless device in response to the medical device being within a communications range of the at least one wireless device; and
upon interrogating the medical device with the wireless device, uploading the data message alert to a remote storage location via the wireless device.

29. The method of claim 28, wherein interrogating the medical device with the wireless device comprises periodically establishing communication with the medical device and reading at least a portion of the memory and wherein uploading the data message alert comprises transmitting the data message alert over a network to at least one of a remote database and the computing device.

30. A computer program product comprising a computer usable medium having control logic stored therein for causing a computer to provide and communicate data message alerts stored on a medical device, the control logic comprising computer readable program code for causing the computer to:
interrogate the medical device; and
upon interrogating the medical device, communicate a data message alert stored in a memory of the medical device wherein the data message alert originates from outside the medical device.

31. The computer program product of claim 30, further comprising computer readable program code for causing the computer to detect whether the data message alert is stored in a dedicated alert field within the memory of the medical device wherein the data message alert is communicated in response to detecting the data message alert stored in the dedicated alert field.

32. The computer program product of claim 31, further comprising computer readable program code for causing the computer to:
receive the data message alert; and
in response to receiving the data message alert, save the data message alert to the dedicated alert field of the medical device.

33. The computer program product of claim 30, further comprising computer readable code for causing the computer to at least one of initialize and install the medical device prior to interrogating the medical device wherein the computer readable code for initializing the medical device includes computer readable code for causing the computer to clear the memory of any data message alerts.

34. The computer program product of claim 30, wherein the computer readable code for causing the computer to communicate the data message alert includes computer readable code for causing the computer to communicate the data message alert in at least one of a variety of data formats compatible for storage in the memory wherein at least one of the data message alert and the variety of data formats compatible for storage in the memory include at least one of the following data formats:
ASCII text;
multi-media;
audio;
audio encoding schema;
XML; and
XML schema definition.

35. A propagated signal on a carrier detectable by a computing system and encoding data and a computer program of instructions for executing a computer process for providing and communicating data message alerts stored on a medical device, the computer process comprising:
receiving a data message alert via a programmer;
in response to receiving the data message alert, saving the data message alert to a memory of the medical device;
interrogating the medical device; and
upon interrogating the medical device, communicating the data message alert via the programmer wherein the data message alert originates from outside the medical device.

36. A system for providing and communicating data message alerts stored on a medical device, the system comprising:
a programmer, a medical device, a link between the programmer and the medical device;
the programmer operative to provide and communicate a data message alert stored on the medical device;
the medical device storing the data message alert in a memory of the medical device; and
the programmer operative to interrogate the medical device and upon interrogating the medical device, communicate the data message alert wherein the data message alert originates from outside the medical device.

37. The system of claim 36, wherein the memory includes a free form data field having the capability to store the data message alert in any data format and wherein the programmer is further operative to communicate the data message alert in any data format in which the data message alert is stored.

38. The system of claim 37, wherein the free form data field comprises a dedicated alert field and wherein the programmer is further operative to detect whether the data message alert is stored in the dedicated alert field and in response to detecting the data message alert stored, communicate the data message alert.

39. The system of claim 36, wherein the link between the programmer and the medical device comprises a radio frequency (RF) signal.

40. The system of claim 38, wherein the programmer includes means for inputting the data message alert and wherein the programmer is further operative to:
receive the data message alert prior to detecting the data message alert stored; and
in response to receiving the data message alert, save the data message alert to the dedicated alert field.

41. The system of claim 36, wherein the programmer includes at least one of a display, a printer, and an audio output device and wherein upon interrogating the medical device, the programmer communicates the data message alert as at least one of the following:
a pop-up window containing an ASCII text message displayed on the display;
a pop-up window containing a multi-media message displayed on the display and played via the audio output device;
an audio message played via the audio output device;
a pop-up window containing an XML text string message displayed on the display; and
a printed text message printed as a header on any printout generated by the programmer until the data message alert is no longer stored in the memory of the medical device.

42. The system of claim 36, wherein the programmer is further operative to persistently communicate the data message alert until the data message alert is acknowledged.

43. The system of claim 36, further comprising a database, and a networked link between the programmer and the database wherein the programmer is further operative to:
upload at least one of the data message alert, associated patient data, and associated medical device data to the database in response to communicating the data message alert stored in the memory; and
in response to uploading, provide verification that at least one of the data message alert, the associated patient data, and the associated medical device data is uploaded to an associated storage location within the database.

44. The system of claim 36, further comprising a wireless device, a link between the wireless device and the medical device and a networked link between the wireless device and the database wherein the wireless device is operative to:
interrogate the medical device in response to the medical device being within a communications range of the wireless device therein detecting whether the data message alert is stored in the memory; and
in response to detecting the data message alert stored in the memory, upload at least one of the data message alert, associated patient data, and associated medical device data to the database.

45. The system of claim 36, further comprising a wireless device, a link between the wireless device and the medical device and a networked link between the wireless device and the programmer wherein the wireless device is operative to:
interrogate the medical device in response to the medical device being within a communications range of the wireless device; and
upon interrogating the medical device, upload the data message alert to the programmer via the networked link.
